(19) United States
(12) Patent Application Publication
(10) Pub. No.: US 2025/0257011 A1
(43) Pub. Date: Aug. 14, 2025

(54) SET OF CHEMICALS FOR THE PRODUCTION OF A CERAMIC DISPERSION OR A CERAMIC GEL

(71) Applicant: CERAMIST One GmbH, Wiesbaden (DE)

(72) Inventor: Dirk MÜLLER, Wiesbaden (DE)

(73) Assignee: CERAMIST One GmbH, Wiesbaden (DE)

(21) Appl. No.: 18/868,868

(22) PCT Filed: May 27, 2023
(86) PCT No.: PCT/EP2023/064303

§ 371 Date: Nov. 25, 2024

(30) Foreign Application Priority Data
May 27, 2022 (DE) 10 2022 113 425.3

(51) Int. Cl. C04B 35/16; B28B 1/00; B33Y 10/00; B33Y 70/10; C04B 35/634; C04B 41/00

(52) U.S. Cl. CPC C04B 35/16; B28B 1/001; B33Y 10/00; B33Y 70/10; C04B 35/63424; C04B 41/0072; C04B 2235/3201; C04B 2235/3205; C04B 2235/3463; C04B 2235/6026; C04B 2235/656

(57) ABSTRACT

The invention relates to a set of chemicals for the production of a ceramic dispersion or a ceramic gel, a process for the production of a ceramic molded part and a ceramic molded part produced by this process.

### FIELD OF THE INVENTION

The invention relates to a set of chemicals for the production of a ceramic dispersion or a ceramic gel, which in turn is suitable for the production of ceramic molded parts such as ceramic veneers or molded bodies such as ceramic partial crowns, veneers, ceramic inlays and onlays, preferably by a layered build-up process such as stereolithography. The invention also relates to a method for producing a ceramic molded part and a ceramic molded part produced by this method.

### BACKGROUND OF THE INVENTION

In a layered build-up process for the production of ceramic veneers or molded bodies, a ceramic gel or a ceramic dispersion with a (curable) polymer material is applied in layers to a base body and dried continuously at the same time or downstream, for example by exposure to radiation or heat. This produces the so-called ceramic “green body”. This is followed by debinding, i.e. the usually polymeric binder is expelled at an elevated temperature, resulting in the so-called “white body”. During debinding, the binder is converted by thermal and/or thermo-chemical processes, the repeating unit of the polymer is at least partially changed and at least partially degraded into volatile components.

The white body is sintered in the sintering furnace during a high-temperature firing. The finely dispersed ceramic powder is compacted and solidified by the effect of temperature, causing the porous component to shrink and increase in strength.

However, layered build-up processes always have the disadvantage that defects such as cracks or bubbles can form during heating, particularly in the debinding step, rendering the component unusable. This effect can usually only be countered by slowing down the heating step, i.e. increasing the duration of the thermal treatment, which, however, makes the corresponding manufacturing process uneconomical.

U.S. Pat. No. 5,496,682, for example, discloses curable compositions for the production of three-dimensional bodies by stereolithography which contain 40 to 70 vol. % of ceramic or metal particles, 10 to 35 wt. % of monomer, 1 to 10 wt. % of photoinitiator, 1 to 10 wt. % of dispersing agent and preferably also solvents, plasticizers and coupling agents.

From EP 2 233 449 A1, slurries for the production of ceramic molded parts by hot-melt inkjet printing processes are known which contain ceramic particles and at least one wax which can be polymerized by free radicals and which produce green bodies which can be debinded essentially without cracking. In stereolithographic processes, however, the slurries must be stable in liquid form over long periods of time, i.e. in particular the particles dispersed in the slurry must not settle prematurely, which poses a particular problem with regard to the desired highest possible volume fraction of ceramic particles in the slurry.

EP 3 147 707 A1 discloses a stereolithography-based process that at least partially overcomes the disadvantages described above. Here too, however, a radical polymer precursor is used to produce the binder.

However, such radical reactions are associated with disadvantages, because the free radicals can damage the other components of the composition, particularly in the case of silicate-based ceramic gels, and thus lead to discoloration. In addition, an exponential increase in the reaction rate is observed in radical reactions at high conversion rates (Trommsdorff-Norrish effect). This effect is due to the decreasing probability of chain termination through recombination of the reactive chain ends, which in turn is caused by the increasing immobility of the growing polymer chains. The resulting increase in the speed of the exothermic reaction causes an increase in temperature, which accelerates the decomposition of the radical initiator and increases the concentration of the reactive molecules. At the same time, the dissipation of the heat of reaction (heat of polymerization) is made more difficult by the increasing viscosity. As a result, localized overheating can occur, which can lead to cracks, explosions or decomposition of the other ingredients of the ceramic composition when applied in layers. The mechanical properties and durability of the ceramic molded parts obtained from the known compositions are also limited, in particular due to their susceptibility to cracking and decomposition. In addition, the ceramic compositions have limited stability/storability.

### AIM

Against this background, the task of the present invention was therefore to provide a set of chemicals with which the above-mentioned disadvantages can be overcome. The set of chemicals according to the invention is intended to enable a complication-free build-up process for silicate mineral-based ceramic gels or ceramic dispersions, from which ceramic molded parts with high quality and low discoloration can be obtained.

### DESCRIPTION OF THE INVENTION

This task is solved according to the invention by a set of chemicals for the production of a ceramic gel, comprising the following components
A) a preferably powdery ceramic material, and
B) a binder, wherein

the ceramic material comprises or consists of a silicate ceramic material selected from the group consisting of feldspar and leucite ceramics and the binder comprises or consists of a gelling agent, wherein the gelling agent is a cross-linked polymer. Preferably, the cross-linked polymer is a polymer having one or more repeating units, wherein at least one of the one or more repeating units has at least one chelating functional group.

The set of chemicals may be suitable for the preparation of a ceramic dispersion, in particular a ceramic suspension and/or a ceramic gel. For this purpose, a solvent and/or dispersing agent such as water or an alcohol can preferably be added to the set of chemicals or the set of chemicals has a corresponding solvent and/or dispersing agent. Preferably, this ceramic dispersion is suitable for use in stereolithographic, binder jetting or material jetting processes.

In colloid chemistry and process engineering, a dispersion is a heterogeneous mixture of at least two substances that do not or hardly dissolve in each other or chemically combine with each other. One or more substances are finely dispersed as a so-called disperse phase in another continuous substance, the so-called dispersion medium.

A “gel” is an example of a disperse system that consists of at least two components. The solid component, the gelling agent, forms a sponge-like, three-dimensional network with its long and/or highly branched molecules, the pores of which are filled with a liquid (lyogel) or a gas (xerogel). The liquid component is thus immobilized in the solid component. If the network is highly porous and air is the embedded gas, the gel is also known as an aerogel.

As scaffolding agents, gelling agents thicken a liquid phase and/or form a rubbery gel. They can therefore also be used as thickening agents or to stabilize emulsions. A distinction is made between natural (e.g. agar-agar), inorganic (e.g. bentonite), semi-synthetic (e.g. carboxymethylcellulose) and synthetic gelling agents (e.g. polyvinyl alcohol). The semi-synthetic and synthetic gelling agents are particularly preferred according to the invention due to their high performance.

According to the invention, “solvent and/or dispersing agent” means a substance which is liquid under standard conditions (temperature: 298.15 K=25° C., pressure: 1013.25 mbar=1013.25 hPa) and in which the components of the set of chemicals can be at least partially dissolved and/or dispersed.

According to the invention, a “binder” is a substance or a combination of substances that enables the production of a stable dispersion or gel by holding and stabilizing the ceramic particles together, in particular during thermal treatment, and enabling uniform distribution. As an essential component, the binder according to the invention comprises or consists of a gelling agent, for example a polymer with a chelating functional group. The binder according to the invention also contributes to the stabilization of the ceramic material in fine distribution in a dissolving and/or dispersing agent, i.e. it makes sedimentation of the ceramic material more difficult or even prevents it completely, provided that a dissolving and/or dispersing agent is used.

In the context of the invention, “polymer” is understood to mean a chemical substance consisting of macromolecules.

“Macromolecules” are molecules that are composed of one or more identical or similar structural units, the constitutional repeating units (IUPAC. Compendium of Chemical Terminology, 2nd ed. (the “Gold Book”), A. D. McNaught, A. Wilkinson, Blackwell Scientific Publications, Oxford (1997), S. J. Chalk. ISBN 0-9678550 Sep. 8). Such macromolecules have more than 10 repeating units, preferably more than 15 repeating units. The molar mass is preferably at least 3,000 g/mol, preferably at least 5,000 g/mol, more preferably at least 7,000 g/mol and most preferably at least 10,000 g/mol.

Polymers are usually produced by the reaction of monomers or oligomers, which have one or more of the constitutional repeating units, in a polymerization reaction. An oligomer is a molecule that has been formed from several monomers and is therefore made up of a large number of structurally identical or similar structural units. Oligomers are referred to in the context of the invention if the molecule was produced from a reaction of 2 to 10, preferably 2 to 8, preferably 3 to 7 monomers.

Preferably, the binder has more than 50 wt. %, preferably more than 70 wt. %, more preferably more than 80 wt. %, even more preferably more than 90 wt. % and most preferably more than 95 wt. % or even 100 wt. % of gelling agent, preferably in the form of a polymer with a chelating functional group.

Preferably, the ceramic material comprises more than 50 wt. %, preferably more than 70 wt. %, more preferably more than 80 wt. %, even more preferably more than 90 wt. % and most preferably more than 95 wt. % of silicate ceramic material.

Silicate ceramic materials are inorganic, non-metallic materials which are obtained from silicate raw materials, i.e. compounds with [SiO₄]⁴-tetrahedra in the crystal structure. The SiO₂ content of the silicate ceramic materials is preferably ≥20 wt. %, more preferably ≥30 wt. %. The term silicate ceramic materials can also include glass ceramics. Preferably, however, these are not included. Preferably, the proportion of glass-ceramics in the silicate ceramic material is less than 5 wt. %, more preferably less than 2 wt. %, even more preferably less than 1 wt. %, even more preferably less than 0.5 wt. % and most preferably less than 0.1 wt. %.

According to the invention, the term “set of chemicals” is understood to mean a predetermined composition of individual chemicals which are present either in separate containers or partially or completely premixed in a composition. Particularly preferably, the set of chemicals is present as a composition, in particular as a liquid, pasty or solid composition.

The term “chelating functional groups” refers to functional groups that are capable of forming chelates. Chelates are cyclic coordination compounds that have at least one central metal atom or ion and a multidentate ligand, the so-called chelating ligand. A ligand is an ion or molecule that can bind, i.e. coordinate, to a central metal atom or ion via a coordinative bond. The difference between the coordinative bond and the classic covalent bond is that the ligand provides both bonding electrons in the coordinative bond; it is therefore a “Lewis base”. Multidentate in this context means that the ligand has two or more coordination sites via which it coordinates to the metal atom or ion. In other words, the chelating ligand must therefore have two or more atomic groupings that can act as electron donors.

In a preferred embodiment of the invention, the chelating ligand has three or more, more preferably four or more, particularly preferably five or more and most preferably six or more coordination sites.

The inventor has observed that by using a gelling agent according to the invention, for example a polymer with a chelating group, a silicate ceramic dispersion or a silicate ceramic gel can be produced which has a high stability of the ceramic particles, i.e. the ceramic particles show no or only a very low tendency to segregate. In addition, the composition has a structure that is easy to process but at the same time sufficiently stable. This was previously not possible or only possible to an insufficient extent.

In particular, when using a polymer with a chelating group, the inventor assumes, without being bound by this theory, that the chelating groups form polar interactions with the partially positively charged silicon atoms of the silicate ceramic and that stabilization, in particular in a dispersion, can be achieved as a result. The polymer material with the at least one chelating functional group thus acts as a binder. Since a corresponding polymer material also increases the viscosity of the gel or dispersion, a flowable and easy-to-process, in particular sprayable, material is obtained. This eliminates the need to use radically polymerizable polymer precursor compounds in production in order to ensure the processability of corresponding ceramic gels.

With the set of chemicals according to the invention, ceramic gels or ceramic dispersions can be produced with which even extremely filigree and small-particle structures can be constructed. In addition, less marginal shrinkage can be observed during the debinding and sintering process, i.e. less pronounced volume changes. The inventor assumes that this is due to the even distribution of the ceramic particles in the gel. In addition, debinding is particularly efficient and fast.

In order to achieve particularly good processability of the gel or dispersion, it preferably has an increased viscosity. In order to achieve this, the gelling agent according to the invention is a cross-linked polymer, more precisely the macromolecules of the polymer of the set of chemicals are cross-linked.

Preferred gelling agents are selected from the group consisting of polysaccharides such as chitosan or chitosan derivatives, alginic acids, xanthan or alginates; polyuronic acids; gelatine; hyaluronic acid; polyvinyl alcohols, polyethylene glycols; ammonium acryloyldimethyltaurate; hydroxypropyl starch (HPS); hydroxypropyl distarch phosphate (HDP); polyquaternium, in particular 3-methyl-1-vinylimidazolium methyl sulphate-N-vinylpyrrolidone copolymer or poly(2-methacryloxyethyltrimethylammonium chloride); bentonites; sorbitan monooleate; polyethylene glycol triether or polypropylene glycol triether; propylene glycol dicaprylate/dicaprate; Silicate clay and/or layered silicates such as sodium-magnesium silicates or aluminium-magnesium silicates, sodium-magnesium or sodium-magnesium-fluorine-lithium layered silicates of the montmorillonite type; polyacrylamide; silicas such as Aerosil; sorbitan oleate decyl glucoside crosspolymers; styrene-maleic anhydride or ethylene-maleic anhydride copolymers and derivatives thereof; hydrophobically modified ethoxylated urethanes; polyvinylpyrrolidone; amylopectin; cellulose or cellulose derivatives such as cellulose acetobutyrate; polyacrylates or polymethacrylates and esters, copolymers and/or salts thereof; or mixtures and copolymers of the foregoing. Preferably, the above-mentioned gelling agents are covalently crosslinked.

The gelling agent is particularly preferably a hydrogel-forming polymer. A hydrogel is a gel made of a water-insoluble polymer that can bind water. The molecules that form the gel are linked chemically, e.g. by covalent, supramolecular or ionic bonds, or physically, e.g. by entanglement of the polymer chains, to form a network that can bind the solvent such as water. Covalently cross-linked polymers are, for example, thiolised polymers (thiomers), which cross-link polymer chains by forming disulphide bridges.

Superabsorbent polymers are a favored special form of gelling agents. Superabsorbent polymers (“superabsorbers”) are preferably cross-linked polymers that are able to absorb many times their own weight in solvents, especially polar solvents such as water. When the liquid is absorbed, the superabsorbent swells and forms a hydrogel. The functioning of superabsorbent polymers is described, for example, in Koltzenburg, S., Nuyken, O., Maskos, M. (2013). Polymers: Synthesis, properties and applications. Germany: Springer Berlin Heidelberg.

According to the invention, the superabsorbent polymer is preferably a crosslinked copolymer with an acrylic acid and/or sodium acrylate and/or acrylic acid ester repeating unit.

According to the invention, the superabsorbent polymer is preferably a crosslinked copolymer with a methacrylic acid and/or sodium methacrylate and/or methacrylic acid ester repeating unit.

Particularly preferred is a cross-linked copolymer of acrylic acid and sodium acrylate or a cross-linked copolymer of acrylic acid and/or sodium acrylate and/or acrylamide, in particular of acrylic acid and sodium acrylate and acrylamide.

Particularly preferred is a crosslinked copolymer of methacrylic acid and sodium methacrylate or a crosslinked copolymer of methacrylic acid and/or sodium methacrylate and/or methacrylamide, in particular of methacrylic acid and sodium methacrylate and methacrylamide. Corresponding copolymers are particularly preferably crosslinked with an allyl ether of sucrose or an aryl ether of pentaerythritol.

Carbomers, in particular those which have been crosslinked with polyalcohols, polyalkenethers such as polyalcohol allyl ethers or polyalkenethers of sugars, allyl ether sucrose or allyl ether pentaerythritol, are particularly preferred-especially due to their wide range of applications and good availability.

Polymers whose chelating group comprises one or more oxygen atoms have also proved to be particularly preferred in use. The inventors assume that the stabilizing effect is particularly high due to the particularly pronounced Si—O interaction. The chelating group can, for example, be an acid, salt or ester group such as in a polyacrylic acid or a polyacrylate. Polyvinylpyrrolidone or polyvinylphosphonates have also proven to be particularly preferred. The macromolecules of the aforementioned polymers are particularly preferably cross-linked, preferably by a covalent cross-linking. The terms “linked” and “cross-linked” are used synonymously in the present case and refer to the linking of a large number of macromolecules of the polymer to form a three-dimensional network. Cross-linking can be achieved directly during the formation of the macromolecules or by reactions on existing polymers. As a result, swelling of the polymer is observed, which in turn has a positive effect on dispersion mediation.

The set of chemicals preferably has less than 5 wt. %, more preferably less than 2 wt. %, even more preferably less than 1 wt. %, still considerably more preferably less than 0.5 wt. % and most preferably less than 0.1 wt. % of polymerizable polymer precursor compounds such as monomers or oligomers.

Particularly preferably, the weight ratio of polymerizable polymer precursor compounds to polymer in the set of chemicals is 1:10 or greater, preferably 1:50 or greater and particularly preferably 1:100 or greater.

A correspondingly low proportion of polymerizable compounds avoids side reactions and decomposition reactions during the production of the gel.

Preferably, the binder and/or the gelling agent has a low ashing residue, preferably ≤10 wt. %, more preferably ≤5 wt. %, even more preferably ≤1 wt. %, considerably more preferably ≤0.5 wt. %, and most preferably ≤0.2 wt. %.

The ashing residue, i.e. the residue remaining after thermal treatment of the ashing, can be determined as described in DIN EN ISO 3451-1, in particular by means of thermogravimetric analysis. A low ashing residue ensures a lower degree of discoloration of the ceramic molded part.

The set of chemicals according to the invention preferably further comprises a solvent and/or dispersing agent. In this context, polar and/or protic solvents such as water and/or alcohols, in particular polyhydric alcohols such as ethylene glycol or monopropylene glycol, are particularly preferred. These are particularly suitable for keeping the components of the gel or dispersion to be obtained from the set of chemicals in dispersed or dissolved form, thereby enabling the gel or dispersion to be processed.

In a preferred embodiment of the invention, the proportion by weight of the solvent and/or dispersing agent in the weight of the set of chemicals is at least 5 wt. %, preferably at least 10 wt. %, more preferably at least 15 wt. %, even more preferably at least 20 wt. %, still considerably more preferably at least 30 wt. % and most preferably at least 40 wt. %, but generally ≤70 wt. %.

Preferably, the proportion by weight of the solvent and/or dispersing agent in the weight of the set of chemicals without including the weight of the ceramic material is 50 wt. % to 99.9 wt. %, more preferably 70 wt. % to 99 wt. % and most preferably 90 wt. % to 99 wt. %.

In a preferred embodiment of the invention, the proportion by weight of the ceramic material in the weight of the set of chemicals is ≤70 wt. %, preferably ≤50 wt. %, more preferably ≤40 wt. %, even more preferably ≤35 wt. % and most preferably ≤30 wt. %, but preferably ≥20 wt. %.

In a preferred embodiment of the invention, the proportion by weight of the ceramic material in the weight of the set of chemicals is in the range of 5-70 wt. %, preferably 10-50 wt. %, even more preferably 15-40 wt. % and most preferably 20-35 wt. %.

By using correspondingly low proportions of ceramic material, it is generally possible to produce a gel that flows well and is thus easy to handle.

Particularly preferably, the composition further comprises one or more bases, in particular a Brønsted base, i.e. a compound whose monomolar aqueous solution has a pH >7, preferably >8. A base ensures that an improved interaction between ceramic particles and polymer material is generated. Particularly in the case of chelating groups with an acid function, the base serves to trap the proton, which further increases the complex formation strength of the chelating group. This effect can be observed in particular with polyacrylic acids or polyacrylates and polyvinylphosphonic acids or polyvinylphosphonates. The base is particularly preferably selected from the group consisting of alkali metal and alkaline earth metal hydroxides and alkali metal and alkaline earth metal carbonates. Particularly preferably, the base is selected from the group consisting of NaOH, KOH, Na₂CO₃, K₂CO₃, CaCO₃, CaO and Ca(OH)₂, NaHCO₃ or mixtures of the foregoing.

The set of chemicals preferably comprises ≤15 wt. %, more preferably less than 10 wt. %, even more preferably ≤5 wt. %, even considerably more preferably ≤1 wt. % and most preferably ≤0.5 wt. % of bases. The minimum content of bases is preferably ≥0.05 wt. %, more preferably ≥0.1 wt. %.

Preferably, the proportion by weight of the base(s) in the weight of the set of chemicals without including the weight of the ceramic material is 0.10 wt. % to 0.99 wt. %, more preferably 0.20wt. % to 0.8 wt. % and most preferably 0.3 wt. % to 0.7 wt. %.

In a preferred embodiment of the invention, the set of chemicals comprises as additional components a preservative which is preferably biocidal. Preservatives serve to increase the shelf life of the ceramic gel or ceramic dispersion, in particular by preventing decomposition processes. Sulfur-containing preservatives, in particular isothiazolinones, are preferably used as preservatives. Due to their bactericidal and fungicidal effect, they protect against microbial decomposition of the components of the set of chemicals. Particularly preferred are 5-chloro-2-methyl-4-isothiazolin-3-ones, 2-methyl-4-isothiazolin-3-ones, isothiazolinone-3-ones, methylisothiazolinone, benzisothiazolinone, octylisothiazolinone, dichloroctylisothiazolinone or mixtures of the aforementioned. Alternatively, formaldehyde releasers such as O-formals or N-formals are also suitable as preservatives. Particularly preferred are benzylhemiformal, 1,6-dihydroxy-2,5-dioxahexane, methylolurea, 7-ethylbicyclooxazolidine, methenamine, paraformaldehyde, tris (hydroxymethyl) nitromethane or mixtures of the foregoing.

The set of chemicals preferably has ≤5 wt. %, more preferably ≤2 wt. %, even more preferably ≤1 wt. %, even considerably more preferably ≤0.5 wt. % and most preferably ≤0.25 wt. % of preservatives. The minimum content of preservatives is preferably ≥0.05 wt. %, more preferably ≥0.1 wt. %.

In order to control the coloring of the ceramic gel or ceramic dispersion, the set of chemicals may further comprise a coloring component, preferably selected from transition metal compounds, in particular oxides such as Er₂O₃, Fe₂O₃, Co₃O₄, MnO₂, NiO₂, Cr₂O₃, Pr₂O₃, Tb₂O₃, Bi₂O₃ and mixtures of the aforementioned. Alternatively, or additionally, however, acetylacetonates or carboxylic acid salts of iron, cerium, praseodymium, nickel, terbium, lanthanum, tungsten, osmium, terbium and manganese can also be used.

The coloring components are preferably selected in such a way that tooth-colored ceramic molding parts can be obtained after debinding and sintering.

The coloring components could preferably also be contained in the ceramic material. The ceramic material may also contain other additives such as flow agents or release agents.

The set of chemicals preferably has less than 5 wt. %, more preferably ≤2 wt. %, even more preferably ≤1 wt. %, even considerably more preferably ≤0.5 wt. % and most preferably ≤0.25 wt. % of the above coloring components. The minimum content of the above coloring components is preferably ≥0.05 wt. %, more preferably ≥0.1 wt. %.

In a preferred embodiment of the invention, the proportion by weight of the ceramic material in the weight of the set of chemicals is at least 40 wt. %, preferably at least 50 wt. %, more preferably at least 55 wt. %, even more preferably at least 60 wt. % and most preferably at least 65 wt. %.

In a preferred embodiment of the invention, the proportion by weight of the ceramic material in the weight of the set of chemicals is at most 95 wt. %, preferably at most 90 wt. %, more preferably at most 85 wt. %, even more preferably at most 80 wt. % and most preferably at most 75 wt. %.

In a preferred embodiment of the invention, the proportion by weight of the ceramic material in the weight of the set of chemicals is in the range of from 50 to 90 wt. %, preferably 55 to 85 wt. %, even more preferably 60 to 80 wt. % and most preferably 65 to 75 wt. %.

A very compact gel with a low ashing residue can generally be obtained by using a correspondingly high proportion of ceramic material.

A correspondingly high proportion of ceramic material also significantly reduces the probability of mechanical damage during thermal treatment of the ceramic gel or dispersion and results in a more homogeneous ceramic molded part.

In a preferred embodiment of the invention, the proportion by weight of the gelling agent, for example in the form of a polymer, in the weight of the set of chemicals is ≥0.1 wt. %, more preferably ≥0.2 wt. %, even more preferably ≥0.5 wt. %, even more preferably at least 1 wt. %, even more preferably at least 1.5 wt. %, even more preferably at least 1.5 wt. %, even more preferably at least 2 wt. %, and most preferably at least 2.5 wt. %. This results in a particularly pronounced stabilization of the ceramic material in the ceramic gel or ceramic dispersion. Preferably, however, the percentage by weight of the polymer is not higher than 3.5 wt. % or 4 wt. %, so that the ashing residue resulting from thermal treatment of the set of chemicals is as low as possible.

Preferably, the weight ratio of the gelling agent in the weight of the set of chemicals without including the weight of the ceramic material is 0.10 wt. % to 0.99 wt. %, more preferably 0.20 wt. % to 0.8 wt. % and most preferably 0.3 wt. % to 0.7 wt. %.

The weight ratio of ceramic material to the weight of the other components of the set of chemicals is preferably in the range of 1:1 to 5:1, more preferably 2:1 to 4:1 and most preferably 3:1 to 4:1. Also highly preferred are ratios of ceramic powder to binder of 60:40 to 70:30.

The silicate ceramic is preferably selected from the group consisting of feldspar, leucite, lithium silicate, lithium disilicate and lithium aluminosilicate ceramics. Feldspar ceramics are particularly preferred, especially in combination with polyacrylic acid and/or a polyacrylate as a polymer. A particularly pronounced stabilizing effect was observed with this combination.

The present invention also relates to a composition, in particular a gel or a dispersion, consisting of or containing the set of chemicals. Preferably, the proportion by weight of the set of chemicals to the total weight of the composition is 10 to 70 wt. %, preferably 15 to 70 wt. %, more preferably 20 to 60 wt. %, even more preferably 25 to 60 wt. % and most preferably 30 to 60 wt. %.

The gel or dispersion is prepared by preferably mechanically mixing component B), i.e. the binder, and optionally contained constituents, such as C) or E), with the ceramic material, which is preferably in the form of a ceramic powder.

The invention also relates to a method for producing a ceramic molded part comprising or consisting of the following steps

a₁) Mixing the components of a set of chemicals as defined above to obtain a gel or dispersion,

b) irradiating or heat treating the gel or dispersion in a temperature range of 50° to 150° C. to obtain a green body,

c) at least partial, preferably complete removal of the binder, preferably by heat treatment of the green body in a temperature range of 150° to 350° C. or by irradiation, to obtain a white body,

d) sintering the white body or, if steps b) and/or c) are optional, the green body or the gel or the dispersion by heat treatment in a temperature range from 700° C. to 1000° C. in order to obtain the ceramic molded part.

The process steps are preferably carried out at different times in the sequence a₁) to d).

Steps b) and c) above are optional and can be carried out together, i.e. in one step without temporal separation. The ceramic molded part can also be produced without the intermediate steps of producing a green body or white body. The binder is then removed in step d). However, a particularly high product quality is obtained when these steps are carried out.

The method according to the invention for producing a ceramic molded part preferably comprises the following additional step:

a₂) layered application of the gel or dispersion to a surface, preferably to a metal or zirconium coping or a mounting die, wherein the application is preferably carried out by spraying and/or a 3D printing process, particularly preferably by means of multi-jet modelling.

This step is preferably carried out between steps a₁) and b) and particularly preferably separated in time from these steps.

Preferably, steps a₂) and/or b) and/or c) and/or d) of the method according to the invention are carried out in a rapid prototyping process, in particular a stereolithography process.

The invention also relates to a ceramic molded part, such as a crown, a crown part, a veneer, a ceramic inlay or onlay, a bridge anchor crown, bridge pontics and parts of the aforementioned, which has been produced by the method according to the invention. By using the set of chemicals according to the invention in the process described above, ceramic molded parts with particularly high homogeneity and natural appearance are obtained. They therefore exhibit preferred properties compared to the ceramic molded parts known from the prior art. The removal of the binder, preferably by heat treatment of the green body in a temperature range of 150° to 350° C. or by irradiation, in particular infrared light irradiation, in order to obtain a white body, is carried out particularly quickly and homogeneously with the gel or dispersion according to the invention. In the above-mentioned temperature range, debinding times of ≤20 minutes, ≤15 minutes or even ≤10 minutes can be achieved.

The ceramic molded parts produced using the method according to the invention are also characterized by their high strength and great attention to detail. The flexural strength according to ISO 6872 is preferably above 50 MPa, in particular in the range of 100 to 500 MPa, for molded bodies made of feldspar or glass ceramics.

The present invention also relates to the use of a combination of a silicate ceramic selected from the group consisting of feldspar and leucite ceramics and a cross-linked polymer in a gel for producing a ceramic veneer.

The invention also relates to the use of a gelling agent as a binder in a gel or dispersion for the manufacture of a ceramic molding, wherein the gel preferably comprises or consists of a silicate ceramic.

The invention also relates to the use of a cross-linked polymer, preferably a polymer having one or more repeating units, more preferably at least one of the repeating units comprising at least one chelating functional group, as a binder in a gel or dispersion for the manufacture of a ceramic veneer, wherein the gel comprises or consists of a silicate ceramic.

### EXAMPLES

The invention will now be described by means of an explicit embodiment example together with corresponding photographic illustrations.

### Production Example 1

A mixture of the following components is prepared in a Petri dish:
1) deionized water (˜70 wt. %)
2) carbomer (˜17 wt. %)
3) Sodium hydroxide (˜3 wt. %)
4) monopropylene glycol (˜7 wt. %)
5) mixture of 5-chloro-2-methyl-4-isothiazolin-3-one, 2-methyl-4-isothiazolin-3-one, isothiazolinone-3-one (remainder)

After obtaining a homogeneous paste, a feldspar or glass ceramic powder is added to this mixture. In relation to the sum of the masses of the mixture with components 1) to 5), 3-4 times the amount of ceramic powder is added in order to obtain a sprayable ceramic dispersion after mechanical mixing. The mixture is transferred to a 10 ml Coltene syringe and applied in layers to a framework body. This is followed by heat treatment at 150° C. to obtain a green body. Subsequent heat treatment at 300° C. produces a white body, which is then fired at 950° C.

### Production Example 2

A mixture of the following components is prepared in a Petri dish:
1) deionized water (˜98.5 wt. %)
2) carbomer (˜1.0 wt. %)
3) potassium hydroxide (˜0.5 wt. %)

After obtaining a homogeneous paste, a feldspar or glass ceramic powder is added to this mixture. In relation to the sum of the masses of the mixture with components 1) to 3), 3-4 times the amount of ceramic powder is added in order to obtain a sprayable ceramic dispersion after mechanical mixing. The mixture is transferred to a 10 ml Coltene syringe and applied in layers to a framework body. This is followed by heat treatment at 150° C. to obtain a green body. Subsequent heat treatment at 300° C. produces a white body, which is then fired at 950° C.

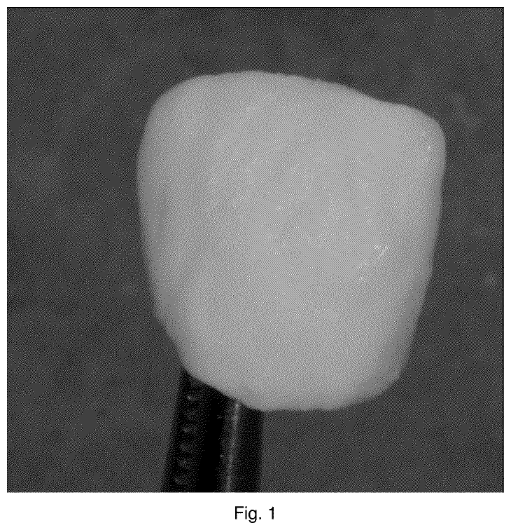

FIG. 1 shows a photographic representation of the white body obtained by the above process “Production example 1” before firing.

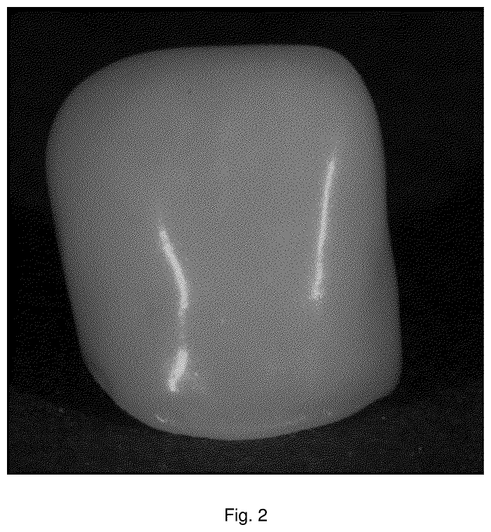

FIG. 2 shows the same white body after firing at 950° C. for 60 min. As can be seen from FIG. 2, a uniform ceramic part with a closed structure is obtained.

1. Set of chemicals, comprising the following components:
a ceramic material comprising or consisting of a silicate ceramic material, and
a binder comprising or consisting of a gelling agent, wherein the gelling agent is a cross-linked polymer,
wherein the silicate ceramic material is selected from the group consisting of feldspar and leucite ceramics.

2. Set of chemicals according to claim 1, wherein the polymer of the gelling agent comprises one or more repeating units, wherein preferably at least one of the one or more repeating units has a chelating functional group.

3. Set of chemicals according to claim 1, wherein the polymer is a superabsorbent polymer, preferably a copolymer with a (meth)acrylic acid and/or sodium (meth)acrylate repeating unit.

4. Set of chemicals according to claim 1, wherein the set comprises the following additional component:
a solvent and/or dispersing agent preferably selected from the group consisting of water and alcohols.

5. Set of chemicals according to claim 1, wherein the set contains the following additional component:
a base, preferably selected from the group consisting of alkali metal and alkaline earth metal hydroxides and alkali metal and alkaline earth metal carbonates.

6. Set of chemicals according to claim 1, wherein the set contains the following additional component:
a preferably biocidal preservative.

7. Set of chemicals according to claim 1, wherein the set contains the following additional component:
a coloring component preferably selected from Er₂O₃, Fe₂O₃, Co₃O₄, MnO₂, NiO₂, Cr₂O₃, Pr₂O₃, Tb₂O₃, Bi₂O₃ and mixtures of the foregoing.

8. Set of chemicals according to claim 1, wherein the weight proportion of the ceramic material in relation to the weight of the set of chemicals is in the range of 50 to 90 wt. %, preferably 60 to 80 wt. %.

9. Gel or dispersion comprising the set of chemicals as defined in any of the preceding claims claim 1.

10. Process for the preparation of a ceramic molded part, comprising the following steps:
a₁) mixing the components of a set of chemicals as defined in any one of the preceding claims to obtain a gel or dispersion,
b) optionally: heat treating the gel or dispersion in a temperature range of from 50° C. to 200° C. to obtain a green body,
c) optionally: removal of the binder by heat treatment of the green body in a temperature range of from 250° C. to 350° C. to obtain a white body, and
d) sintering by heat treatment in a temperature range of from 700° C. to 1000° C. to obtain the ceramic molded part.

11. Process of manufacturing a ceramic molded part according to claim 10, comprising the following additional step:
a₂) layered application of the gel or dispersion to a surface, preferably by spraying and/or a 3D printing process, particularly preferably by binder jetting, material jetting, poly-jet or multi-jet modeling.

12. Ceramic molded part obtainable by a process as defined in claim 10.

13. Use of a combination of a silicate ceramic material selected from the group consisting of feldspar and leucite ceramics and a cross-linked polymer in a gel or a dispersion, for the production of a ceramic veneer.
